(19) United States
(12) Patent Application Publication
(10) Pub. No.: US 2025/0359545 A1
(43) Pub. Date: Nov. 27, 2025

(54) Anti-pathogenic aerosol compositions

(71) Applicant: SQUIRE KENT PTY LIMITED, Crows Nest (AU)

(72) Inventors: Peter Yassa, Crows Nest (AU); Mark Willcox, Crows Nest (AU); Rajesh Kuppusamy, Crows Nest (AU)

(21) Appl. No.: 18/841,977

(22) PCT Filed: Feb. 27, 2023
(86) PCT No.: PCT/AU2023/050130

§ 371 Date: Aug. 27, 2024

(30) Foreign Application Priority Data
Feb. 28, 2022 (AU) 2022900472

(51) Int. Cl. A01N 25/06; A01N 31/02; A01N 33/12; A01N 47/44; A01P 1/00

(52) U.S. Cl. CPC A01N 25/06; A01N 31/02; A01N 33/12; A01N 47/44; A01P 1/00

(57) ABSTRACT

Disclosed herein is an anti-pathogenic aerosol composition comprising an alcohol selected from ethanol and isopropanol; an alkyldimethylbenzylammonium compound; polyhexanide; and water.

### CROSS-REFERENCE TO RELATED APPLICATIONS

The present application claims priority to AU 2022900472, the entire disclosure of which is incorporated herein by cross-reference.

### FIELD OF THE DISCLOSURE

The present disclosure broadly relates to anti-pathogenic aerosol compositions and the use thereof.

### BACKGROUND OF THE DISCLOSURE

Any discussion of the prior art throughout this specification should in no way be considered as an admission that such prior art is widely known or forms part of the common general knowledge in the field.

Pathogens such as bacteria and viruses pose a significant threat to human health. Enclosed areas, such as rooms, provide a fertile environment for humans to become infected with contagious pathogens, such as viruses. As such, effective cleaning/decontamination of enclosed and partly enclosed spaces is an important tool in the fight against the transmission of contagious pathogens. The importance of preventing the spread of such pathogens has been a key focus in the fight against the Covid19 pandemic.

Against this background the present inventors have developed anti-pathogenic aerosol compositions and methods for dispensing them.

### SUMMARY OF THE DISCLOSURE

In a first aspect there is provided an aerosol composition comprising:
a) an alcohol selected from ethanol and isopropanol;
b) an alkyldimethylbenzylammonium compound;
c) polyhexanide; and
d) water.

The following options may be used in conjunction with the first aspect, either individually or in any combination.

The alcohol may be present in the composition in an amount between about 0.5% and about 20% (v/v), or in an amount between about 1% and about 20% (v/v), or in an amount between about 1% and about 15% (v/V).

The alkyldimethylbenzylammonium compound may be present in the composition in an amount between about 0.001% and about 5% (w/v), or in an amount between about 0.001% and about 1% (w/v), or in an amount between about 0.005% and about 1% (w/v), or in an amount between about 0.01% and about 1% (w/v), or in an amount between about 0.1% and about 1% (w/v), or in an amount between about 0.1% and about 0.8% (w/v), or in an amount between about 0.5% and about 1% (w/V).

The polyhexanide may be present in the composition in an amount between about 0.005% and about 10% (w/v), or in an amount between about 0.005% and about 5% (w/v), or in an amount between about 0.05% and about 5% (w/v), or in an amount between about 0.05% and about 3% (w/v), or in an amount between about 0.5% and about 3% (w/v), or in an amount between about 0.5% and about 2.5% (w/v), or in an amount between about 1% and 3% (w/V).

The water may be present in the composition in an amount between about 10% and about 95% (v/v), or in an amount between about 15% and about 95% (v/v), or in an amount between about 15% and about 90% (v/v).

In one embodiment, the alcohol is ethanol.

The alkyldimethylbenzylammonium compound may have the following formula (I):

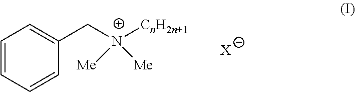

wherein n is an integer between 6 and 20 and X is a halide.

In one embodiment the alkyldimethylbenzylammonium compound is benzyldimethylhexadecylammonium chloride.

The polyhexanide may have an average molecular weight between about 3000 Da and about 3600 Da.

The composition may further comprise a propellant.

The propellant may be dimethyl ether.

The composition may be a liquid.

The composition may be a self-propellant composition.

The composition may be a single phase composition.

The composition may be an anti-pathogenic composition.

The composition may be a viricidal composition.

The composition may be a bactericidal composition.

The composition may be housed in a can.

When applied, the composition may leave no, or substantially no residue.

The composition may comprise an alkyldimethylbenzylammonium compound and polyhexanide as the sole active ingredients.

The composition may not contain any glycol compounds.

In a second aspect there is provided a method for killing or inactivating a pathogen in a space, the method comprising dispensing a composition of the first aspect as an aerosol into the space.

The following options may be used in conjunction with the second aspect, either individually or in any combination.

The pathogen may be an airborne pathogen.

The pathogen may be suspended in air.

The pathogen may be on a surface within the space.

The pathogen may be killed or inactivated in air within the space.

The pathogen may be killed or inactivated on a surface within the space.

The surface may be a horizontal surface.

The pathogen may be a bacteria, a virus, mold or fungi.

The pathogen may be a virus.

The virus may be an enveloped virus.

The enveloped virus may be a coronavirus, a herpes virus or an influenza virus.

The coronavirus may be 229E, NL63, OC43, HKU1, SARS-CoV-2, SARS-CoV or MERS-CoV.

The bacteria may be E. Coli, Legionella, Bacillus subtilis or Staphylococcus aureus.

The space may be an enclosed space or a partly enclosed space.

The aerosol may have a droplet size of less than 10 microns, or less than 5 microns.

The aerosol may be dispensed by a fogging device.

The aerosol may be dispensed from a spray can.

### Definitions

The following are some definitions that may be helpful in understanding the description of the present disclosure. These are intended as general definitions and should in no way limit the scope of the present disclosure to those terms alone, but are put forth for a better understanding of the following description.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The terms “a” and “an” are used herein to refer to one or to more than one (i.e. to at least one) of the grammatical object of the article. By way of example, “an element” means one element or more than one element.

In the context of this specification the term “about” is understood to refer to a range of numbers that a person of skill in the art would consider equivalent to the recited value in the context of achieving the same function or result.

In the context of this specification the term “aerosol composition” is understood to mean a composition that is suitable for dispensing as an aerosol. In other words, the term “aerosol composition” is synonymous with the term “a composition suitable for dispensing as an aerosol”.

In the context of this specification the term “halide” is understood to mean chloro, fluoro, bromo or iodo.

Any numerical range recited herein is intended to include all sub-ranges of the same numerical precision subsumed within the recited range. For example, a range of 1.0 to 5.0 is intended to include all sub-ranges between (and including) the recited minimum value of 1.0 and the recited maximum value of 5.0, that is, having a minimum value equal to or greater than 1.0 and a maximum value equal to or less than 5.0, such as 2.1 to 4.5. Any maximum numerical limitation recited herein is intended to include all lower numerical limitations subsumed therein and any minimum numerical limitation recited herein is intended to include all higher numerical limitations subsumed therein.

### DETAILED DESCRIPTION

In one aspect of the disclosure there is provided an aerosol composition comprising:
a) an alcohol selected from ethanol and isopropanol;
b) an alkyldimethylbenzylammonium compound;
c) polyhexanide; and
d) water.

In one embodiment the alkyldimethylbenzylammonium compound has the following formula (I):

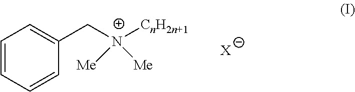

wherein n is an integer between 6 and 20 and X is a halide.

In another embodiment n is an integer between 8 and 18. In a further embodiment n is 8, 10, 12, 14, 16 or 18. In yet another embodiment, n is 16. In some embodiments X is chloro, bromo or iodo. In other embodiments X is chloro. The composition may comprise multiple alkyldimethylbenzylammonium compounds of the formula (I). For example, in one embodiment the composition comprises a plurality of compounds of formula (I) in which n is 8, 10, 12, 14, 16 and 18. In a further embodiment, the alkyldimethylbenzylammonium compound is benzyldimethylhexadecylammonium chloride (also known as cetalkonium chloride).

The alkyldimethylbenzylammonium compound may be present in the composition in an amount between about 0.001% and about 4% (w/v), or in an amount between about 0.005% and about 3% (w/v), or in an amount between about 0.01% and about 3% (w/v), or in an amount between about 0.01% and 2% (w/v), or in an amount between about 0.01% and about 1% (w/v), or in an amount between about 0.001% and about 3% (w/v), or in an amount between about 0.001% and about 2% (w/v), or in an amount between about 0.001% and about 1% (w/v), or in an amount between about 0.02% and about 1% (w/v), or in an amount between about 0.02% and about 0.85% (w/V).

The alkyldimethylbenzylammonium compound may alternatively be present in the composition in an amount between about 0.001% and about 5% (w/v), or in an amount between about 0.001% and about 1% (w/v), or in an amount between about 0.005% and about 1% (w/v), or in an amount between about 0.01% and about 1% (w/v), or in an amount between about 0.1% and about 1% (w/v), or in an amount between about 0.1% and about 0.8% (w/V), or in an amount between about 0.5% and about 1% (w/v). The alkyldimethylbenzylammonium compound may be present in the composition in an amount between about 0.1% and about 1% (w/v). The alkyldimethylbenzylammonium compound may be present in the composition in an amount of about 0.6% (w/V).

Polyhexanide (also known as polyhexamethylenebiguanide) has the following structure:

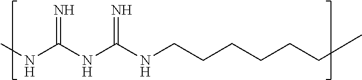

In some embodiments the polyhexanide has an average molecular weight between about 500 Da and about 50,000 Da, or an average molecular weight between about 1,000 Da and about 10,000 Da, or an average molecular weight between about 2,500 Da and about 4,000 Da, or an average molecular weight between about 3,000 Da and about 3,600 Da. The polyhexanide may have an average molecular weight between about 3,000 Da and about 3,600 Da.

The polyhexanide may be present in the composition in an amount between about 0.001% and about 5% (w/v), or in an amount between about 0.001% and about 4% (w/V), or in an amount between about 0.001% and about 3% (w/v), or in an amount between about 0.005% and about 5% (w/v), or in an amount between about 0.005% and about 4% (w/v), or in an amount between about 0.005% and about 3% (w/v), or in an amount between about 0.01% and about 5% (w/v), or in an amount between about 0.01% and about 4% (w/v), or in an amount between about 0.01% and about 3% (w/v), or in an amount between about 0.02% and about 3% (w/v), or in an amount between about 0.02% and about 2.75% (w/V).

The polyhexanide may alternatively be present in the composition in an amount between about 0.05% and about 5% (w/v), or in an amount between about 0.05% and about 3% (w/v), or in an amount between about 0.5% and about 3% (w/v), or in an amount between about 0.5% and about 2.5% (w/v), or in an amount between about 1% and 3% (w/V). The polyhexanide may be present in the composition in an amount between about 1% and about 3% (w/v). The polyhexanide may be present in the composition in an amount of about 2% (w/v).

The water may be present in the composition in an amount between about 10% and about 95% (v/v), or in an amount between about 10% and about 90% (v/v), or in an amount between about 15% and about 90% (v/v), or in an amount between about 18% and about 90% (v/v), or in an amount between about 30% and about 90% (v/v), or in an amount between about 50% and about 90% (v/v), or in an amount between about 70% and about 90% (v/v), or in an amount between about 80% and about 90% (v/v). The water may be present in the composition in an amount between about 80% and about 90% (v/v). The water may be present in the composition in an amount between about 85% and about 90% (v/v). The water may be present in the composition in an amount of about 90% (v/V).

The alcohol may be present in the composition in an amount between about 0.5% and about 20% (v/v), or in an amount between about 1% and about 20% (v/v), or in an amount between about 10% and about 20% (v/v), or in an amount between about 1% and about 15% (v/v), or in an amount between about 2% and about 15% (v/v), or in an amount between about 2% and about 10% (v/v). The alcohol may be present in the composition in an amount between about 10% and about 20% (v/v). The alcohol may be present in the composition in an amount between about 10% and about 15% (v/v). The alcohol may be present in the composition in an amount of about 10% (v/v).

In one embodiment, the composition further comprises a propellant. Propellants suitable for use in the composition include, but are not limited to: liquefied petroleum gas (such as ethane, propane, ethylene, isobutane, n-butane, propylene and the like), chlorofluorocarbons, fluorocarbons, nitrogen gas, carbon dioxide gas and dimethyl ether. In one embodiment, the propellant is dimethyl ether.

The propellant may be present in the composition in an amount between about 60% and about 90% (v/v), or in an amount between about 65% and about 90% (v/v), or in an amount between about 65% and about 85% (v/v), or in an amount between about 65% and about 75% (v/v), or in an amount between about 65% and about 80% (v/v), or in an amount between about 70% and about 85% (v/v), or in an amount between about 75% and about 85% (v/v). The propellant may be present in the composition in an amount between about 60% and about 90% (v/v). The propellant may be present in the composition in an amount of about 70% (v/v).

In embodiments where the composition comprises a propellant:
The alcohol may be present in the composition in an amount between about 0.5% and about 10% (v/v), or in an amount between about 0.5% and about 5% (v/v), or in an amount between about 2% and about 3% (v/v), or in an amount between about 1% and about 4% (v/v), or in an amount between about 1% and about 3% (v/v). The alcohol may be present in the composition in an amount between about 1% and about 4% (v/v). The alcohol may be present in the composition in an amount between about 3% and about 5% (v/v). The alcohol may be present in the composition in an amount of about 3% (v/V).
The alkyldimethylbenzylammonium compound may be present in the composition in an amount between about 0.001% and about 5% (w/v), or in an amount between about 0.001% and about 3% (w/v), or in an amount between about 0.005% and about 3% (w/v), or in an amount between about 0.01% and about 2% (w/v), or in an amount between about 0.01% and about 1% (w/v), or in an amount between about 0.1% and about 2% (w/v), or in an amount between about 0.1% and about 0.8% (w/v), or in an amount between about 0.5% and about 1% (w/v). The alkyldimethylbenzylammonium compound may be present in the composition in an amount between about 0.1% and about 1% (w/v). The alkyldimethylbenzylammonium compound may be present in the composition in an amount of about 0.6% (w/V).
The polyhexanide may be present in the composition in an amount between about 0.005% and about 5% (w/v), or in an amount between about 0.01% and about 5% (w/v), or in an amount between about 0.01% and about 2% (w/v), or in an amount between about 0.05% and about 4% (w/v), or in an amount between about 0.5% and about 3.5% (w/v), or in an amount between about 1% and about 4% (w/v), or in an amount between about 0.5% and about 2.5% (w/v), or in an amount between about 1% and 3% (w/v). The polyhexanide may be present in the composition in an amount between about 1% and about 3% (w/v). The polyhexanide may be present in the composition in an amount of about 2% (w/V).
The water may be present in the composition in an amount between about 10% and about 35% (v/v), or in an amount between about 15% and about 35% (v/v), or in an amount between about 10% and about 30% (w/v), or in an amount between about 20% and about 30% (w/v), or in an amount between about 10% and about 25% (v/v), or in an amount between about 15% and about 20% (v/v). The water may be present in the composition in an amount between about 20% and about 30% (w/v). The water may be present in the composition in an amount between about 25% and about 27% (w/V). The water may be present in the composition in an amount between about 27% (w/V).

The composition may further comprise one or more fragrances, for example natural fragrances. Natural fragrances include naturally derived oils such as oil of Bergamot, Bitter Orange, Lemon, Mandarin, Caraway, Cedar Leaf, Clove Leaf, Cedar Wood, Geranium, Lavender, Orange, Origanum, Petitgrain, White Cedar, Patchouli, Lavandin, Neroli, Rose absolute, and the like. Natural perfumes include the extracts of blossoms, stems and leaves, fruits, fruit peel, roots, woods, herbs and grasses, needles and branches, resins and balsams. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetivert oil, olibanum oil, galbanum oil, ladanum oil and lavendin oil. In some embodiments the composition may further comprise cinnamaldehyde.

In some embodiments the compositions may comprise propylene glycol and/or potassium sorbate.

In some embodiments the composition may also comprise a corrosion inhibitor. When the composition is stored in a metal container such as a metal can, the corrosion inhibitor prevents the metal from corroding. Suitable corrosion inhibitors include octadecyldiethanolamide, aminomethyl propanol benzoate, amine salts of mono- and di-mixed isooctyl phosphate dodecyl ester, ammonium benzoate salts such as ammonium benzoate, dodecyl benzoate ammonium, and tetradecyl benzoate ammonium, butyl lauroyl sarcosinate, nitromethane, morpholine, hexamethylene imine, piperidine and ammonia. The composition may comprise a corrosion inhibitor which is ammonium benzoate. The corrosion inhibitor may be present in the composition in an amount between about 0.01 and about 1% (w/v), or between about 0.01% and about 0.1%, about 0.01% and about 0.25%, about 0.01% and about 0.5%, about 0.01% and about 0.75%, about 0.1% and about 0.25%, about 0.1% and about 0.5%, about 0.1% and about 0.75%, about 0.1% and about 1%, about 0.25% and about 0.5%, about 0.1% and about 0.75%, about 0.1% and about 1%, about 0.5% and about 0.75%, about 0.5% and about 1%, or between about 0.75% and about 1% (w/v). The corrosion inhibitor may be present in the composition in an amount of about 0.2% (w/V). The corrosion inhibitor may be ammonium benzoate, present in the composition in an amount of about 0.2% (w/v). Where the composition comprises a propellant, the corrosion inhibitor may be present in the composition in an amount between about 0.01 and about 0.1% w/v, or about 0.01% and about 0.05%, or about 0.05% and about 0.1%, or about 0.02 and about 0.07% (w/v). The corrosion inhibitor may be present in the composition in an amount of about 0.06% (w/V).

In some embodiments, the composition comprises an alkyldimethylbenzylammonium compound and polyhexanide as the sole active, i.e. anti-pathogenic, ingredients. In the context of this specification, water and an alcohol selected from ethanol and isopropanol are not considered to be active, i.e. anti-pathogenic ingredients. In the present composition water serves as a diluent and the alcohol assists in aerosolization of the composition.

In some embodiments, the composition does not contain any glycol compounds. Glycol compounds are understood to be compounds having two hydroxyl groups attached to adjacent carbon atoms, such as propylene glycol and triethylene glycol.

In one embodiment, the composition comprises:
a) ethanol in an amount between about 1% and about 5% (v/V);
b) benzyldimethylhexadecylammonium chloride in an amount between about 0.05% and about 3% (w/V);
c) polyhexanide in an amount between about 0.5% and about 5% (w/v);
d) a propellant in an amount between about 65% and about 85% (v/v); and
e) water in an amount between about 10% and about 25% (v/V).

In another embodiment, the composition comprises:
a) ethanol in an amount between about 1% and about 3.5% (v/v);
b) benzyldimethylhexadecylammonium chloride in an amount between about 0.05% and about 2% (w/V);
c) polyhexanide in an amount between about 0.5% and about 3% (w/V);
d) a propellant in an amount between about 75% and about 85% (v/v); and
e) water in an amount between about 15% and about 20% (v/V).

In yet another embodiment, the composition comprises:
a) ethanol in an amount between about 1% and about 3% (v/V);
b) benzyldimethylhexadecylammonium chloride in an amount between about 0.1% and about 1% (w/V);
c) polyhexanide in an amount between about 1% and about 3% (w/V);
d) a propellant in an amount between about 75% and about 85% (v/v); and
e) water in an amount between about 16% and about 20% (v/V).

In still a further embodiment, the composition comprises:
a) ethanol in an amount of about 2% (v/V);
b) benzyldimethylhexadecylammonium chloride in an amount of about 0.6% (w/V);
c) polyhexanide in an amount of about 2% (w/V);
d) a propellant in an amount of about 80% (v/v); and
e) water in an amount of about 18% (v/v).

In one embodiment, the composition comprises:
a) ethanol in an amount between about 1% and about 5% (v/V);
b) benzyldimethylhexadecylammonium chloride in an amount between about 0.05% and about 3.5% (w/V);
c) polyhexanide in an amount between about 0.5% and about 5% (w/v);
d) a propellant in an amount between about 60% and about 80% (v/v); and
e) water in an amount between about 10% and about 25% (v/V).

In another embodiment, the composition comprises:
a) ethanol in an amount between about 1% and about 4% (v/V);
b) benzyldimethylhexadecylammonium chloride in an amount between about 0.05% and about 2% (w/V);
c) polyhexanide in an amount between about 0.5% and about 3.5% (w/V);
d) a propellant in an amount between about 65% and about 80% (v/v); and
e) water in an amount between about 10% and about 20% (v/V).

In yet another embodiment, the composition comprises:
a) ethanol in an amount between about 1% and about 3% (v/V);
b) benzyldimethylhexadecylammonium chloride in an amount between about 0.1% and about 1.5% (w/V);
c) polyhexanide in an amount between about 1% and about 3% (w/V);
d) a propellant in an amount between about 68% and about 82% (v/v); and
e) water in an amount between about 16% and about 20% (v/V).

In still a further embodiment, the composition comprises:
a) ethanol in an amount of about 3% (v/V);
b) benzyldimethylhexadecylammonium chloride in an amount of about 0.6% (w/V);
c) polyhexanide in an amount of about 2% (w/V);
d) a propellant in an amount of about 70% (v/v); and
e) water in an amount of about 27% (v/V).

In yet another embodiment, the composition comprises:
a) ethanol in an amount between about 2% and about 3% (v/v);
b) benzyldimethylhexadecylammonium chloride in an amount between about 0.03% and about 0.18% (w/V);
c) polyhexanide in an amount between about 0.03% and about 0.6% (w/V);
d) a propellant in an amount between about 70% and about 80% (v/v); and
e) water in an amount between about 17% and about 27% (v/V).

In one embodiment, the composition comprises:
a) ethanol in an amount between about 1% and about 5% (v/V);
b) benzyldimethylhexadecylammonium chloride in an amount between about 0.05% and about 3% (w/V);
c) polyhexanide in an amount between about 0.5% and about 5% (w/V);
d) a propellant in an amount between about 60% and about 85% (v/v); and
e) water in an amount between about 15% and about 35% (v/V).

In another embodiment, the composition comprises:
a) ethanol in an amount between about 2% and about 4% (v/V);
b) benzyldimethylhexadecylammonium chloride in an amount between about 0.05% and about 2% (w/v);
c) polyhexanide in an amount between about 0.5% and about 3% (w/V);
d) a propellant in an amount between about 65% and about 80% (v/v); and
e) water in an amount between about 15% and about 30% (v/v).

In yet another embodiment, the composition comprises:
a) ethanol in an amount between about 2% and about 4% (v/V);
b) benzyldimethylhexadecylammonium chloride in an amount between about 0.1% and about 1% (w/V);
c) polyhexanide in an amount between about 1% and about 3% (w/V);
d) a propellant in an amount between about 65% and about 75% (v/v); and
e) water in an amount between about 20% and about 30% (v/v).

In yet another embodiment, the composition comprises:
a) ethanol in an amount between about 2.5% and about 3.5% (v/V);
b) benzyldimethylhexadecylammonium chloride in an amount between about 0.1% and about 1% (w/V);
c) polyhexanide in an amount between about 1.5% and about 2.5% (w/V);
d) a propellant in an amount between about 65% and about 75% (v/v); and
e) water in an amount between about 20% and about 30% (v/V).

In yet another embodiment, the composition comprises:
a) ethanol in an amount between about 2.5% and about 3.5% (v/V);
b) benzyldimethylhexadecylammonium chloride in an amount between about 0.1% and about 1% (w/V);
c) polyhexanide in an amount between about 1.75% and about 2.25% (w/V);
d) a propellant in an amount between about 65% and about 75% (v/v); and
e) water in an amount between about 25% and about 30% (v/v).

In yet another embodiment, the composition comprises:
a) ethanol in an amount between about 1% and about 4% (v/V);
b) benzyldimethylhexadecylammonium chloride in an amount between about 0.01% and about 2% (w/V);
c) polyhexanide in an amount between about 0.01% and about 3% (w/V);
d) a propellant in an amount between about 65% and about 85% (v/v); and
e) water in an amount between about 15% and about 30% (v/v).

In yet another embodiment, the composition comprises:
a) ethanol in an amount between about 1% and about 4% (v/V);
b) benzyldimethylhexadecylammonium chloride in an amount between about 0.01% and about 2% (w/v);
c) polyhexanide in an amount between about 0.01% and about 3% (w/v);
d) a propellant in an amount between about 70% and about 80% (v/v); and
e) water in an amount between about 20% and about 30% (v/v).

In yet another embodiment, the composition comprises:
a) ethanol in an amount between about 0.1% and about 6% (v/V);
b) benzyldimethylhexadecylammonium chloride in an amount between about 0.05% and about 0.25% (w/V);
c) polyhexanide in an amount between about 0.2% and about 1% (w/V);
d) a propellant in an amount between about 60% and about 90% (v/V);
e) ammonium benzoate in an amount between about 0.02% and about 0.08% (w/V);
and
f) water in an amount between about 8% and about 36% (v/v).

In yet another embodiment, the composition comprises:
a) ethanol in an amount between about 3% and about 4.5% (v/V);
b) benzyldimethylhexadecylammonium chloride in an amount of about 0.18% (w/V);
c) polyhexanide in an amount of about 0.6% (w/V);
d) a propellant in an amount of about 70% (v/V);
e) ammonium benzoate in an amount of about 0.06% (w/v); and
f) water in an amount between about 25% and about 27% (v/V).

In yet another embodiment, the composition consists of:
a) an alcohol selected from ethanol and isopropanol;
b) an alkyldimethylbenzylammonium compound;
c) polyhexanide; and
e) water.

In yet another embodiment, the composition consists of:
a) an alcohol selected from ethanol and isopropanol;
b) an alkyldimethylbenzylammonium compound;
c) polyhexanide;
e) a corrosion inhibitor; and
e) water.

In yet another embodiment, the composition consists of:
a) ethanol in an amount between about 10% and about 15% (w/V);
b) benzyldimethylhexadecylammonium chloride in an amount of about 0.6% (w/V);
c) polyhexanide in an amount of about 2% (w/V);
e) ammonium benzoate in an amount of about 0.2% (w/v); and
e) water in an amount of about 85% and about 90% (w/V).

In yet another embodiment, the composition consists of:
a) an alcohol selected from ethanol and isopropanol;
b) an alkyldimethylbenzylammonium compound;
c) polyhexanide;
d) a propellant; and
e) water.

In yet another embodiment, the composition consists of:
a) an alcohol selected from ethanol and isopropanol;
b) an alkyldimethylbenzylammonium compound;
c) polyhexanide;
d) a propellant;
e) a corrosion inhibitor; and
f) water.

In yet another embodiment, the composition consists of:
a) an alcohol selected from ethanol and isopropanol;
b) an alkyldimethylbenzylammonium compound;
c) polyhexanide;
d) a propellant;
e) a corrosion inhibitor;
f) fragrance; and
g) water.

In yet another embodiment, the composition consists essentially of:
a) an alcohol selected from ethanol and isopropanol;
b) an alkyldimethylbenzylammonium compound;
c) polyhexanide; and
e) water.

In yet another embodiment, the composition consists essentially of:
a) an alcohol selected from ethanol and isopropanol;
b) an alkyldimethylbenzylammonium compound;
c) polyhexanide;
e) a corrosion inhibitor; and
e) water.

In yet another embodiment, the composition consists essentially of:
a) ethanol in an amount between about 10% and about 15% (w/V);
b) benzyldimethylhexadecylammonium chloride in an amount of about 0.6% (w/V);
c) polyhexanide in an amount of about 2% (w/V);
e) ammonium benzoate in an amount of about 0.2% (w/v); and
e) water in an amount of about 85% and about 90% (w/V).

In yet another embodiment, the composition consists essentially of:
a) an alcohol selected from ethanol and isopropanol;
b) an alkyldimethylbenzylammonium compound;
c) polyhexanide;
d) a propellant; and
e) water.

In yet another embodiment, the composition consists essentially of:
a) an alcohol selected from ethanol and isopropanol;
b) an alkyldimethylbenzylammonium compound;
c) polyhexanide;
d) a propellant;
e) a corrosion inhibitor; and
f) water.

In yet another embodiment, the composition consists essentially of:
a) an alcohol selected from ethanol and isopropanol;
b) an alkyldimethylbenzylammonium compound;
c) polyhexanide;
d) a propellant;
e) a corrosion inhibitor;
f) fragrance; and
g) water.

In the embodiments in paragraphs to above, the propellant may be dimethyl ether.

In the embodiments in paragraphs to above, the amounts and characteristics of the alcohol, alkyldimethylbenzylammonium compound, and polyhexanide may be as set out in each of the embodiments of paragraphs to [0071].

In some embodiments the aerosol may have a droplet size of less than 20 microns, or less than 18 microns, or less than 16 microns, or less than 14 microns, or less than 12 microns, or less than 10 microns, or less than 8 microns, or less than 6 microns, or less than 5 microns, or less than 4 microns, or less than 3 microns, or less than 2 microns, or between about 0.05 microns and about 10 microns, or between about 0.05 microns and about 8 microns, or between about 0.05 microns and about 6 microns, or between about 0.05 microns and about 5 microns, or between about 0.1 microns and about 5 microns, or between about 0.5 microns and about 5 microns, or between about 1 micron and about 5 microns, or between about 0.05 microns and about 4 microns, or between about 0.1 microns and about 4 microns, or between about 0.5 microns and about 4 microns, or between about 1 micron and about 4 microns, or between about 0.1 microns and about 3 microns, or between about 0.5 microns and about 3 microns, or between about 1 micron and about 3 microns. The droplet size may be an average droplet size.

The compositions may be prepared by methods familiar to those skilled in the art. For example, compositions comprising an alcohol, an alkyldimethylbenzylammonium compound, polyhexanide and water may be prepared by admixing the desired amounts of each component followed by stirring until a homogenous mixture is obtained. Compositions comprising a propellant may be prepared by adding a mixture of an alcohol, an alkyldimethylbenzylammonium compound, polyhexanide and water in the desired amounts to a container (30% by volume), followed by addition of a propellant (70% by volume). The container is then sealed and fitted with an appropriate dispenser suitable for dispensing the composition as an aerosol. Preferably the container is a can.

The compositions find use in anti-pathogenic applications, for example as bactericides and/or viricides. Accordingly, in another aspect of the disclosure there is provided a method for killing or inactivating a pathogen in a space, the method comprising dispensing a composition of the disclosure as an aerosol into the space.

Any standard form of commercial aerosol dispenser may be employed to dispense the compositions, including but not limited to: aluminium and tin-plated steel containers, optionally comprising coatings and/or internal container linings, for example resin coatings such as epoxy resin resins. In one embodiment the compositions are dispensed using a spray can. For example, one or more spray cans may be placed in various positions in a room. The can(s) are then activated to release the aerosol composition. After a period of about 5-10 minutes the contents of the can(s) are emptied into the room resulting in the killing/inactivating of any pathogens present. This method of dispensing is analogous to so-called “cockroach bombs” used for killing cockroaches in enclosed and partly enclosed spaces. The aerosol dispenser may comprise between about 150 mL and about 500 mL, or between about 250 mL and about 500 mL of the composition.

Alternatively, the compositions may be provided in a form suitable for dispensing using a fogging device. Examples of fogging devices include thermal foggers, electric foggers, pressurized air foggers, ultrasonic foggers and low energy vaporizers. In embodiments where a fogging device is used, a propellant is not required in the compositions. Preferably, the fogging device is capable of dispensing the compositions where the droplet size of the aerosol is less than about 4 microns.

Spaces in which pathogens may be killed/inactivated include any and all spaces whether enclosed or partly enclosed. Suitable spaces include, but are not limited to, a room, a shipping container, a hospital ward, an aircraft interior, a car interior, a bus interior, a train interior, a house, an aged care facility, a gym, an ambulance, a taxi, a ride-share vehicle, corporate offices or meeting rooms, a shopping mall, an elevator, a subway system or a warehouse. Other suitable spaces will be recognised by those skilled in the art.

The method may be used to kill/inactivate pathogens including bacteria, viruses, mold and fungi. Surprisingly, the inventors have found that the compositions are capable of killing/inactivating viruses in air (i.e viruses which are airborne, i.e. suspended in the air). That is, aerosolised particles of the composition which are suspended or floating in the air can contact and inactivate viruses which are also suspended or floating in the air. The compositions are also capable of killing/inactivating bacteria present on surfaces. Inactivating a virus is understood to mean that following contact with a composition of the disclosure the virus is rendered unable to infect a host.

In some embodiments the virus is an enveloped virus, such as for example a coronavirus, a herpes virus or an influenza virus.

Coronaviruses constitute the subfamily Orthocoronavirinae. They are enveloped viruses with a positive-sense single-stranded RNA genome and a nucleocapsid of helical symmetry. Coronaviruses cause respiratory tract infections in humans that range from mild to lethal. Non-limiting examples of coronaviruses include 229E, NL63, OC43, HKU1, SARS-CoV-2, SARS-CoV or MERS-CoV. Other coronaviruses will be apparent to those skilled in the art.

The influenza virus may be influenza A, B, C or D.

The fungi may be Candida albicans or Aspergillus brasiliensis.

Non-limiting examples of bacteria include E. Coli, Legionella, Staphylococcus aureus, Salmonella typhimurium, Enterococcus hirae, Acinetobacter baumanii, Pseudomonas aeruginosa, Campylobacter jejuni, Bacillus subtillis, Listeria monocytogenes and mycobacteria.

### EXAMPLES

### Example 1

Prior to aerosolisation a composition comprising 85:15 water:ethanol containing 5% (w/V) polyhexanide (average molecular weight 3000-3600 Da) and 0.6% (w/V) benzyldimethylhexadecylammonium chloride was tested for its ability to prevent the replication of MHV-1 in mouse A9 ATCC/CCL 1.4 cells by using the protocol outlined in https://doi.org/10.1016/j.clae.2021.101513. Briefly, viruses, at 1×10⁶ plaque forming units (PFU)/mL as the final concentration were incubated in the composition for 20 minutes.

A9 cells were seeded in 12-well tissue culture plates at 5-10×10⁵ cells per well and allowed to adhere overnight at 37° C. in 5% CO₂. After incubation, the culture medium was removed and cells were washed with 1× phosphate buffered saline. Thereafter, 100 μL of serially diluted (in Dey Engley neutralising broth; Becton Dickinson and Company, Macquarie Park, NSW, Australia) viruses in the composition were inoculated in each well and incubated for 20 min at ambient temperature. After incubation, the cells were overlaid with 1% agarose (Sigma-Aldrich, Castle Hill, NSW, Australia) and DMEM solution (1:1) and further incubated for 72 h. Following incubation, the cells were fixed with 4% formaldehyde for 1.5-2 h. The number of viral plaque forming units was quantified after staining cells with 1% crystal violet (Sigma-Aldrich). Reduction in PFU compared to the negative control (PBS) was calculated. The results are shown below.

| | Dilution | Well | Well | Well | Aver- | Total | Log₁₀ |
|---|---|---|---|---|---|---|---|
| | factor | 1 | 2 | 3 | age | PFU/mL | reduction |
| Control | 5 | 14 | 18 | 15 | 16 | 1.57 × 10⁶ | — |
| Test | 0 | 0 | 0 | 0 | 0 | 0 | >5.2 |
| composi- | | | | | | | |
| tion | | | | | | | |

The test composition completely killed the SARS-CoV-2 coronavirus surrogate MHV-1 giving at least a 5 log₁₀ reduction in viral number.

### Example 2

Prior to aerosolisation the following compositions were tested for activity against Bacillus subtilis spores.

| Component | A | B | C | D |
|---|---|---|---|---|
| EtOH (%) | 8 | 16 | 10 | 10 |
| Water (%) | 92 | 84 | 90 | 90 |
| polyhexanide (mg) | 2.5 | 5.0 | 2.5 | 25 |
| BDMHDAC\hat{ } (mg) | 1 | 2 | 6 | 6 |

\hat{ }Benzyldimethylhexadecylammonium chloride

B. subtilis spores were prepared by inoculating Bacillus subtilis ATCC 19659 in Tryptic soy broth (TSB) and incubating overnight at 37° C. for 12-16 hours. Following incubation, 100 mL of bacterial suspension was plated on Nutrient agar (NA; supplemented with 500 ppm MnSO₄) and incubated at 37° C. for 10-14 days to stimulate the production of spores. Spores were harvested by washing the agar surface with sterile deionized chilled water. After washing with sterile deionized chilled water, spores were stored in sterile deionized water at 4° C. and used in future experiments.

To test the activity of compositions A to D against the B. subtilis spores, the spores were resuspended in water to an OD₆₆₀ of 0.102. 100 μL of spore suspension was added to 100 μL of disinfectant solution in duplicate. For controls, 100 μL of spore suspension was added to 100 μL of water. These suspensions were then incubated at ambient temperature (21° C.) for 20 minutes. After incubation, 100 μL of solution was added to 900 μL of Dey Engley neutralizing broth (Oxoid, Basingstoke, UK) and serially diluted. Aliquots (20 μL) were then added onto TSB agar plates and incubated at 35° C. for 24 h. After incubation, the number of bacterial colonies were counted. The results are shown below.

| | | | | | | Reduction |
|---|---|---|---|---|---|---|
| | | | | | | compared |
| | | | | | Total | to control |
| | Dilution | Plate | Plate | | colony | (log₁₀ |
| Composition | factor | 1 | 2 | Average | count | CFU) |
| control | 3 | 16 | 15 | 16 | 775000 | — |
| A | 0 | 0 | 0 | 0 | 0 | ≥5 |
| B | 0 | 0 | 0 | 0 | 0 | ≥5 |
| C | 0 | 0 | 0 | 0 | 0 | ≥5 |
| D | 0 | 0 | 0 | 0 | 0 | ≥5 |

As the spores of B. subtilis are known to be highly resistant to disinfectants, the fact that the compositions were active against the spores indicates that they are also likely to be active against cells of other bacteria, such as staphylococci and Pseudomonas.

### Example 3

A composition in accordance with one embodiment of the disclosure was tested for its ability to kill an airborne coronavirus. The composition comprised the following components: 0.6% w/v benzyldimethylhexadecylammonium chloride, 2% w/v polyhexanide (average molecular weight 3000-3600 Da), 70 mL of dimethyl ether, 27 ml of water and 3 mL of ethanol. This composition is referred to below as “Aerocide C19 Bomb”.

Prior to commencing the tests several standards were evaluated to determine whether they disclosed suitable protocols to test the ability of aerosolised compositions to kill aerosolised viruses. ISO 16000:36 “Indoor air—Part 36: Standard method for assessing the reduction rate of culturable airborne bacteria by air purifiers using a test chamber” and EN17272:2020 “Chemical disinfectants and antiseptics-Methods of airborne room disinfection by automated process-Determination of bactericidal, mycobactericidal, sporicidal, fungicidal, yeasticidal, virucidal and phagocidal activities” were located. However, neither standard disclosed a suitable protocol.

ISO 16000:36 discloses methods designed to evaluate the effectiveness of “electrically-powered device that is basically built of a fan and a set of components possessing the ability to capture and/or (partially or totally) destroy air pollutants”. As such, it does not evaluate the activity of aerosolised disinfectants.

EN17272:2020 discloses methods for “assessing the disinfectant activity of airborne surface disinfection processes”, whereby microbes are dried onto stainless steel disks which are then placed in a closed chamber within which the disks are placed. It is evident that this standard evaluates an aerosolised disinfectant, but only its ability to reduce microbial numbers on surfaces, not microbial numbers in the air.

As a result, aspects of both ISO 16000:36 and EN17272:2020 were utilised to develop a new protocol that allowed aerosolisation of anti-pathogenic compositions and microbes and then capture of the microbes after a period of time in which the anti-pathogenic compositions and microbes were present together as aerosols within a sealed container.

### Experimental Protocol

The Aerocide C19 Bomb was tested against airborne mouse coronavirus ATCC/VR261 (MHV-1) in a sealable 3 m³ chamber. The experiments were conducted at ambient laboratory temperature set at 21° C., and ambient laboratory humidity. The virus was aerosolized using a nebulizer from Tekceleo that generates 12 μm aerosols.

### Cleaning of the Experimental Apparatus

The working chamber and the nebulizer were cleaned with 0.5% hydrogen peroxide and then with 10 ml of 1× phosphate buffered saline (PBS) for 3 minutes to ensure that the instrument was clean without any traces of hydrogen peroxide.

### Mouse Coronavirus

For controls, the viral suspension (1×10⁵ plaque forming units) in Dulbecco's Modified Eagle Medium was aerosolized inside the tent for 4 min. After 4 minutes the air vapour droplets were collected by vacuum pumping through an Anderson sieve onto four glass Petri dishes containing 2% w/v agar (BD, Macquarie Park, NSW, Australia). The Anderson sieve collects the largest (4.7 μm) sized aerosols at the top and the smallest (1.1 μm) at the bottom of the device.

For the test samples, the viral suspension (1×10⁵ plaque forming units) in Dulbecco's Modified Eagle Medium (DMEM; Thermofisher, Macquarie Park, NSW, Australia) was aerosolized inside the tent for 4 min at the same time as an aerosol can containing the Aerocide C19 Bomb (manufactured by Moorebank Aerosol) was activated into the sealed chamber for 4 min. After 4 min, the air vapour droplets were collected using the Anderson sieve device as above.

The control and test samples were removed from the agar by scrapping into 1500 μL of 20% w/v bovine serum albumin for each plate. A9 mouse fibroblast cells (ATCC/CCL 1.4), that are permissive for the growth of the mouse coronavirus, were grown to confluence in DMEM containing 10% foetal bovine serum (FBS; Thermofisher), 100 μg/ml streptomycin sulfate and 100 I.U. penicillin G, (Thermofisher). Aliquots (100 L) of the collected viral particles from each agar plate were inoculated into the wells of 12 well plates containing A9 cells and incubated for 1 hour at 37° C. in the presence of 5% (v/v) CO₂. The plates were gently rocked once every 15 minutes to prevent the cells from drying out. After incubation, an overlay media containing a 50:50 mix of 2% (w/v) agar (Sigma-Aldrich, Castle Hill, NSW, Australia) and DMEM was added to each well and further incubated for 72 hours. Following incubation, the cells were fixed with 4% (v/v) formaldehyde (Sigma-Aldrich) for 2-3 hours, the agar overlay removed and the number of plaques produced by viral particles (plaque-forming units; pfu) visualized after staining with 1% (w/V) crystal violet (Sigma-Aldrich).

### Results

| | Plate Number/ | | Total viral cells (pfu) |
|---|---|---|---|
| Sample | aerosol size (μm) | Total count | (log₁₀) |
| Control with no | 1/4.7 | 30 | 203 (2.3) |
| Aerocide C19 | 2/3.3 | 60 | |
| Bomb | 3/2.1 | 113 | |
| | 4/1.1 | 0 | |
| Test with | 1/4.7 | 0 | 0 |
| Aerocide C19 | 2/3.3 | 0 | |
| Bomb | 3/2.1 | 0 | |
| | 4/1.1 | 0 | |

### Conclusion

Under the test conditions, the Aerocide C19 Bomb reduced the numbers of aerosolised mouse coronavirus by >2 log 10 pfu. The Aerocide C19 Bomb is therefore effective in killing airborne coronavirus.

### Example 4

The Aerocide C19 Bomb used in Example 3 above was tested for its ability to kill bacteria.

The protocol followed that of the efficacy test of EN17272:2020, but with the following modifications:
1. S. aureus ATCC 6538 and E. coli ATCC 10798 were used
2. 0.3% Bovine Serum Albumin (BSA) was used
3 Glass slides were used
4. A sealed chamber of 3 m³ was used
5. The distribution test was not performed
6. Preliminary test to validate absence of residual effect (efficacy test and distribution test) was not performed
7. The carriers were placed horizontally and vertically in the test chamber
8. Ramasol (Quin Global Pty Ltd) was used as a comparative control.

### Bacteria Suspensions

Bacteria were initially grown on tryptone soy agar (TSA) and then in tryptone soy broth (TSB). After overnight growth at 35° C., the bacteria were centrifuged and the turbidity was adjusted so that OD₆₆₀ nm was 0.1. The bacterial suspension was then further diluted in 0.3% BSA to achieve 1×10⁶ CFU ml-1 as a final bacterial test suspension. The bacterial concentration was confirmed by plating serial dilutions in D/E neutralising solution onto TSA.

### Testing

The test suspensions of bacteria were utilized within 2 h of preparation. 50 μL of test suspensions were spread on glass slides and dried in a bio-safety cabinet for 45 minutes. Once the solution had dried, the glass slides were placed in the front and back of a 3 m³ sealable chamber in horizontal and vertical positions at not below 50% of the tent height.

An aerosol can comprising the C19 Aerosol Bomb was triggered and the chamber was closed whilst the Aerocide C19 Bomb was active for 4 minutes. After 20 minutes, the tent was opened and the glass slides recovered.

Glass slides were transferred to 10 mL of D/E neutralising solution and 5 sterile glass beads added. The tubes are vortexed for 1 minute and then the solution was removed and 10-fold serially diluted in D/E neutralising solution.

Aliquots (100 ml) of the solutions were then added onto TSA and incubated at 35° C. for 24 h. After incubation, the number of bacterial colonies were counted.

### Results

The results are shown below in Tables 1 and 2.

TABLE 1
Activity of Aerocide C19 Bomb against surfaces contaminated with bacteria

| | | | | | Log₁₀ | |
|---|---|---|---|---|---|---|
| | | | | | reduction | |
| | Initial | Test | | Bacteria | compared | Average |
| Test | suspension | Solution | Carrier position | remaining | to control | log₁₀ |
| Organism | titre | titre | and test validation | on carrier | carrier | reduction |
| Gram | | | | | | |
| positive | | | | | | |
| bacterium | | | | | | |
| S. aureus | 1.3 × 10⁸ | 8 × 10⁶ | Control Carrier | 7.3 × 10⁶ | 0 | — |
| ATCC | | | Horizontal front-1 | 0 | ≥6 | ≥6 |
| 6538 | | | Horizontal back-1 | 0 | ≥6 | |
| | | | Horizontal front-2 | 0 | ≥6 | |
| | | | Horizontal back-2 | 0 | ≥6 | |
| | | | Vertical front-left | 5.2 × 10⁵ | 1.15 | 1.9 |
| | | | Vertical- front right | 0 | ≥6 | |
| | | | Vertical back-left | 3.9 × 10⁶ | 0.27 | |
| | | | Vertical back-right | 4.3 × 10⁶ | 0.23 | |
| Gram | | | | | | |
| negative | | | | | | |
| bacterium | | | | | | |
| E. coli | 1.6 × 10⁸ | 1 × 10⁶ | Control carrier | 1.2 × 10⁶ | 0 | — |
| ATCC | | | Horizontal front-1 | 0 | ≥6 | ≥6 |
| 10798 | | | Horizontal front-2 | 0 | ≥6 | |
| | | | Horizontal back-1 | 0 | ≥6 | |
| | | | Horizontal back-2 | 0 | ≥6 | |
| | | | Vertical front-left | 1.6 × 10⁶ | −0.13 | −0.008 |
| | | | Vertical front-right | 0.9 × 10⁶ | 0.13 | |
| | | | Vertical back-left | 1.1 × 10⁶ | 0.04 | |
| | | | Vertical back-right | 1.4 × 10⁶ | −0.07 | |

TABLE 2
Activity of Ramasol (control) against surfaces contaminated with bacteria

| | | | | | Log₁₀ | |
|---|---|---|---|---|---|---|
| | | | | | reduction | |
| | Initial | Test | | Bacteria | compared | Average |
| Test | suspension | Solution | Carrier position | remaining | to control | log₁₀ |
| Organism | titre | titre | and test validation | on carrier | carrier | reduction |
| Gram | | | | | | |
| positive | | | | | | |
| bacterium | | | | | | |
| S. aureus | 2.1 × 10⁸ | 1 × 10⁷ | Control carrier | 1.2 × 10⁷ | 0 | — |
| ATCC | | | Horizontal front-1 | 1.1 × 10⁴ | 3.04 | 4.1 |
| 6538 | | | Horizontal back-1 | 5.6 × 10³ | 3.33 | |
| | | | Horizontal front-2 | 8 × 10³ | 3.18 | |
| | | | Horizontal front-2 | 0 | ≥7 | |
| | | | Vertical front-left | 0 | ≥7 | 1.8 |
| | | | Vertical front-right | 1.2 × 10⁷ | 0 | |
| | | | Vertical back-left | 1.2 × 10⁷ | 0 | |
| | | | Vertical back-right | 1.2 × 10⁷ | 0 | |

### Conclusion

Under the test conditions, the Aerocide C19 Bomb provided good disinfectant activity against microorganisms on horizontal surfaces and meets the >5 log 10 reduction of EN 17272:2020. The Ramasol aerosols were not able to meet the requirements of EN 17272:2020.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of an two or more of said steps, features, compositions and compounds.

1.-30. (canceled)

31. A method for killing or inactivating a virus suspended in air in a space, the method comprising dispensing an aerosol composition into the space, the aerosol composition comprising:
a) an alcohol selected from ethanol and isopropanol;
b) an alkyldimethylbenzylammonium compound;
c) polyhexanide; and
d) water.

32. The method of claim 31, wherein the alcohol is present in the composition in an amount between about 0.5% and about 20% (v/v).

33. The method of claim 31, wherein the alkyldimethylbenzylammonium compound is present in the composition in an amount between about 0.001% and about 5% (w/v).

34. The method of claim 31, wherein the polyhexanide is present in the composition in an amount between about 0.005% and about 10% (w/v).

35. The method of claim 31, wherein the water is present in the composition in an amount between about 10% and about 95% (v/v).

36. The method of claim 31, wherein the alcohol is ethanol.

37. The method of claim 31, wherein the alkyldimethylbenzylammonium compound has the following formula (I): 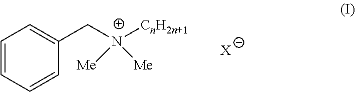
wherein n is an integer between 6 and 20 and X is a halide.

38. The method of claim 37, wherein the alkyldimethylbenzylammonium compound is benzyldimethylhexadecylammonium chloride.

39. The method of claim 31, wherein the polyhexanide has an average molecular weight between about 3000 Da and about 3600 Da.

40. The method of claim 31, wherein the composition further comprises a propellant.

41.-43. (canceled)

44. The method of claim 31, wherein the virus is an enveloped virus.

45. The method of claim 44, wherein the enveloped virus is a coronavirus, a herpes virus or an influenza virus.

46. The method of claim 44, wherein the enveloped virus is a coronavirus.

47. The method of claim 46, wherein the coronavirus is 229E, NL63, OC43, HKU1, SARS-CoV-2, SARS-CoV or MERS-CoV.

48. The method of claim 46, wherein the coronavirus is SARS-CoV-2.

49. The method of claim 31, wherein the space is an enclosed space or a partly enclosed space.

50. The method of claim 31, wherein the aerosol composition has a droplet size of less than 10 microns, or less than 5 microns.

51. The method of claim 31, wherein the aerosol composition is dispensed by a fogging device or from a spray can.

52. (canceled)

53. The method of claim 31, wherein the aerosol composition does not contain any glycol compounds.

54. The method of claim 31, wherein the aerosol composition consists of:
a) an alcohol selected from ethanol and isopropanol;
b) an alkyldimethylbenzylammonium compound;
c) polyhexanide;
d) optionally a corrosion inhibitor;
e) optionally a propellant; and
f) water.
